(19) United States
(12) Patent Application Publication
(10) Pub. No.: US 2014/0086857 A1
(43) Pub. Date: Mar. 27, 2014

(54) Compositions Comprising Siloxane Polymer

(71) Applicant: John D. Blizzard, Bay City, MI (US)

(72) Inventor: John D. Blizzard, Bay City, MI (US)

(73) Assignee: QUADSIL, INC., Midland, MI (US)

(21) Appl. No.: 14/115,276

(22) PCT Filed: May 10, 2012
(86) PCT No.: PCT/US12/37264

§ 371 Date: Nov. 1, 2013

Related U.S. Application Data
(60) Provisional application No. 61/518,933, filed on May 13, 2011.

(51) Int. Cl. C09D 5/14; A61K 31/165; A61K 31/235; A61K 31/045; A61K 31/133; A61Q 17/04; A61Q 19/00; A61Q 15/00; A61Q 19/08; A61Q 5/12; A61Q 5/04; A61Q 3/02; A61Q 1/02; A61Q 1/10; A01N 25/10; A61K 8/89; A61Q 5/02; A61Q 5/06; A61Q 9/04; A61Q 9/02; A61Q 1/14; A61Q 1/12; A61Q 1/06; A61Q 1/08; A61Q 19/10; A61K 47/34; C09D 7/12

(52) U.S. Cl. CPC C09D 5/14; C09D 7/1233; A61K 31/165; A61K 31/235; A61K 31/045; A61K 31/133; A61Q 17/04; A61Q 19/00; A61Q 15/00; A61Q 19/08; A61Q 5/12; A61Q 5/04; A61Q 3/02; A61Q 1/02; A61Q 1/10; A01N 25/10; A61K 8/89; A61Q 5/02; A61Q 5/06; A61Q 9/04; A61Q 9/02; A61Q 1/14; A61Q 1/12; A61Q 1/06; A61Q 1/08; A61Q 19/10; A61Q 19/007; A61Q 19/002; A61K 47/34
USPC 424/59; 524/268; 523/122; 514/627; 514/544; 514/729; 514/669; 424/65; 424/61; 424/70.12; 424/73; 424/69; 424/64; 424/63

(57) ABSTRACT

Compositions comprising siloxane polymers are disclosed. The compositions are useful as thixotropic bases for a variety of components, such as antimicrobial agents, sterilants, medicaments, antibiotics, analgesics, essential oils, preservatives, colorants, fragrances, and the like. Methods for preparing the compositions are also disclosed.

### CROSS REFERENCE TO RELATED APPLICATIONS

This application claims the benefit of U.S. Application No. 61/518,933 filed May 13, 2011, the entire disclosure of which is incorporated herein by reference.

### BACKGROUND OF THE INVENTION

The material of this invention is a siloxane polymer thixotropic base that is useful as an excipient for many actives or variable materials that can be compounded into it. For purposes of this invention, “excipient” is used in a broader sense than simply carrying pharmaceuticals. “Actives” for purposes of this invention means any material that will provide a benefit to a user, such as medicaments (e.g., triple antibiotic), pharmaceuticals (e.g., acetylsalicylic acid), cosmetics (e.g., colorants for the skin), and the like.

U.S. Pat. No. 5,654,362, which is incorporated herein in its entirety, discloses that a process for producing a pre-precursor gel is carried out by reacting an ≡Si—H containing polysiloxane with an α,ω-diene in the presence of platinum catalyst and a low molecular weight silicone oil, wherein a gel is formed. Thereafter, the precursor is formed by subjecting the gel to additional amounts of low molecular oil under a shearing force to form a paste.

The precursor paste is easily formed and handled and can be used in cosmetic preparations and other such materials, but it has a drag and resistance to spreading into a smooth layer, when applied to the skin. After application, the paste has a tacky touch, which in some cases can be repellent to the cosmetic user.

What is desirable is a thixotropic base suitable for delivering components topically to the user without undesirable feel. The invention is directed to these, as well as other, important ends.

### SUMMARY OF THE INVENTION

Accordingly, in one embodiment, the invention is directed to compositions, comprising:

A. a siloxane gel comprising:
a. a first solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom;
b. a siloxane copolymer comprising a reaction product of:
   i. an hydridopolysiloxane selected from the group consisting of:
      (a) R₃SiO(R′₂SiO)ₐ(R″HSiO)_{b}SiR₃;
      (b) HR₂SiO(R′₂SiO)_{c}SiR₂H; and
      (c) HR₂SiO(R′₂SiO)ₐ(R″HSiO)_{b}SiR₂H;
      wherein:
         R, R′, R″ are, each independently, C₁-C₆alkyl;
         a is about 0 to about 250;
         b is about 1 to about 250; and
         c is about 0 to about 250; and
   ii. an α,ω-diene having the general formula:

CH₂═CH(CH₂)ₓCH═CH₂
wherein:
   x is about 1 to about 20;

B. a first additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom;

C. a second additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom.

In another embodiment, the invention is directed to methods of preparing a siloxane polymer thixotropic base, comprising:
reacting to form a copolymer:
i. a ≡Si—H containing polysiloxane selected from the group consisting of:
   (a) R₃SiO(R₂SiO)ₐ(R″HSiO)_{b}SiR₃;
   (b) HR₂SiO(R′₂SiO)_{c}SiR₂H; and
   (c) HR₂SiO(R₂SiO)ₐ(R″HSiO)_{b}SiR₂H;
      wherein:
      R, R′, R″ are, each independently, C₁-C₆alkyl;
      a is about 0 to about 250;
      b is about 1 to about 250; and
      c is about 0 to about 250; and
ii. an α,ω-diene having the general formula:

CH₂═CH(CH₂)ₓCH═CH₂
wherein:
   x is about 1 to about 20;
wherein said reaction is carried out in the presence of a Noble metal catalyst and a first solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom; and
wherein said reaction is continued until a gel is formed by crosslinking and addition of said ≡Si—H across double bonds in said alpha, omega-diene;
adding a first additional solvent to said gel and subjecting said first additional solvent and said gel to shear force until a siloxane paste is formed;
wherein said first additional solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom;
adding a second additional solvent to said siloxane paste and blending said second additional solvent and said siloxane paste by mixing until said siloxane polymer thixotropic base is formed;
wherein said second additional solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom.

In certain embodiments, additional components may be added to said siloxane gel, said siloxane paste, or said siloxane polymer thixotropic base. Such components include antimicrobial agents, sterilants, medicaments, antibiotics, analgesics, essential oils, preservatives, colorants, fragrances, and mixtures thereof.

### DETAILED DESCRIPTION OF THE INVENTION

As employed above and throughout the disclosure, the following terms, unless otherwise indicated, shall be understood to have the following meanings.

As used herein, the singular forms “a,” “an,” and “the” include the plural reference unless the context clearly indicates otherwise.

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

Accordingly, in one embodiment, the invention is directed to compositions, comprising:

A. a siloxane gel comprising:
a. a first solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom;
b. a siloxane copolymer comprising a reaction product of:
   i. an hydridopolysiloxane (≡Si—H containing) polysiloxane selected from the group consisting of:
      (a) R₃SiO(R₂SiO)ₐ(R″HSiO)_{b}SiR₃;
      (b) HR₂SiO(R′₂SiO)_{c}SiR₂H; and
      (c) HR₂SiO(R′₂SiO)ₐ(R″HSiO)_{b}SiR₂H;
      wherein:
         R, R′, R″ are, each independently, C₁-C₆alkyl;
         a is about 0 to about 250;
         b is about 1 to about 250; and
         c is about 0 to about 250; and
   ii. an α,ω-diene having the general formula:

CH₂═CH(CH₂)ₓCH═CH₂
wherein:
   x is about 1 to about 20;

B. a first additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom;

C. a second additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom.

Some representative examples of suitable α,ω-diene for use herein are 1,4-pentadiene; 1,5-hexadiene; 1,6-heptadiene; 1,7-octadiene; 1,8-nonadiene; 1,9-decadiene; 1,11-dodecadiene; 1,13-tetradecadiene; and 1,19-eicosadiene.

The addition and crosslinking reaction requires a catalyst to effect the reaction between the ≡SiH containing polysiloxane and the α,ω-diene. Suitable catalysts are Group VIII transition metals, i.e., the Noble metals. Such Noble metal catalysts are described in U.S. Pat. No. 3,923,705, incorporated herein by reference, to show platinum catalysts. One preferred platinum catalyst is Karstedt's catalyst, which is described in U.S. Pat. No. 3,715,334 and U.S. Pat. No. 3,814,730, incorporated herein by reference. Karstedt's catalyst is a platinum divinyl tetramethyl disiloxane complex typically containing about one weight percent of platinum in a solvent such as toluene. Another preferred platinum catalyst is a reaction product of chloroplatinic acid and an organosilicon compound containing terminal aliphatic unsaturation. It is described in U.S. Pat. No. 3,419,593, incorporated herein by reference. The Noble metal catalysts are used in amounts from about 0.00001 to about 0.5 parts per 110 weight parts of the ≡SiH containing polysiloxane, preferable about 0.00001 to about 0.02 parts, most preferable is about 0.0001 to about 0.002 parts.

The ≡Si—H containing polysiloxane are commercially available and are described hereinafter.

The phrase “low molecular weight silicone oil” is intended herein to include low molecular weight linear and cyclic volatile methyl siloxanes, low molecular weight linear and cyclic volatile and non-volatile alkyl and aryl siloxanes, and low molecular weight linear and cyclic functional siloxanes. Most preferred, however, are low molecular weight linear and cyclic volatile methyl siloxanes (VMS).

Volatile methyl siloxanes correspond to the average unit formula (CH₃)ₐSiO_{(4-a)/2} in which a has an average value of two to three. The compounds contain siloxane units joined by Si—O—Si bonds. Representative units are monofunctional “M” units (CH₃)₃SiO_{1/2} and difunctional “D” units (CH₃)₃SiO_{2/2}.

The presence of trifunctional “T” units CH₃SiO_{3/2} results in the formation of branched linear or cyclic volatile methylsiloxanes. The presence of tetrafunctional “Q” units SiO_{4/2} units results in the formation of branched linear or cyclic volatile methyl siloxanes.

Linear volatile methyl siloxanes have the formula (CH₃)₃SiO{(CH₃)₂SiO}_{y}Si (CH₃)₃. The value of y is 0 to 5. Cyclic volatile methyl siloxanes have the formula {(CH₃)₂SiO}ₓ. The value of z is 3 to 6. Preferable, these volatile methyl siloxanes have boiling points less than about 250° C. and viscosities of about 0.65 to 5.0 centistokes (mm²/s).

Representative linear polysiloxanes are compounds of the formula R₃SiO(R₂SiO)_{y}SiR₃, and representative cyclic polysiloxanes are compounds of the formula (R₂SiO)_{z}. R is an alkyl group of 1 to 6 carbon atoms, or an aryl group such as phenyl. The value of y is 0 to 80, preferably 0 to 20. The value of z is 0 to 9, preferably 4 to 6. These polysiloxanes have viscosities generally in the range of about 1 to 100 centistokes (mm²/s).

The invention herein is not limited to swelling silicone elastomers with only low molecular weight polysiloxanes. Other types of solvents can swell the silicone elastomer. Thus, a single solvent or a mixture of solvents may be used.

By solvent we mean organic compounds, compounds containing a silicon atom, mixtures of organic compounds, mixtures of compounds containing a silicon atom, or mixtures of organic compounds and compounds containing a silicon atom, used on an industrial scale to dissolve, suspend, or change the physical properties of other materials.

In general, the organic compounds are aromatic hydrocarbons, aliphatic hydrocarbons, alcohols, aldehydes, ketones, amines, esters, ethers, glycols, glycol ethers, alkyl halides, or aromatic halides. Representative of some common organic solvents are alcohols such as methanol, ethanol, i-propanol, cyclohexanol, benzyl alcohol, 2-octanol, ethylene glycol, propylene glycol, and glycerol; aliphatic hydrocarbons such as pentane, cyclohexane, heptanes, VM&P solvent, and mineral spirits; alkyl halides such as ethyl chloride, and chlorobenzene; amines such as isopropylamine, cyclohexylamine, ethanolamine, and diethanolamine; aromatic hydrocarbons such as benzene, toluene, ethyl benzene, and xylene; esters such as ethyl acetate, isopropyl acetate, ethyl acetoacetate, amyl acetate, isobutyl isobutyrate, and benzyl acetate; ethers such ethyl ether, n-butyl ether, tetrahydrofuran, and 1,4-dioxane; glycol ethers such as ethylene glycol monomethyl ether, ethylene glycol monomethyl ether acetate, diethyleneglycol monobutyl ether, and propylene glycol monophenyl ether; ketones such as acetone, methyl ethyl ketone, cyclohexanone, diacetone alcohol, methyl amyl ketone, and diisobutyl ketone; petroleum hydrocarbons such as mineral oil, gasoline, naptha, kerosene, gas oil, heavy oil, and crude oil; lubricating oils such as spindle oil and turbine oil; and fatty oils such as corn oil, soybean oil, olive oil, rape seed oil, cotton seed oil, sardine oil, herring oil and whale oil.

Other miscellaneous organic solvents can also be used such as acetonitrile, nitromethane, dimethylforamide, propylene oxide, trioctyl phosphate, butyrolactone, furfural, pine oil, turpentine, and m-creosol.

It is also intended herein to encompass by the term “solvent”, volatile flavoring agents such as oil of wintergreen; peppermint oil; spearmint oil; menthol; vanilla; cinnamon oil; clove oil; bay oil; anise oil, eucalyptus oil; thyme oil; cedar leaf oil; oil of nutmeg; oil of sage; cassia oil; cocoa; licorice; high fructose corn syrup; citrus oils such as lemon, orange, lime and grapefruit; fruit essences such as apple, pear, peach, grape, strawberry, raspberry, cherry, plum, pineapple, and apricot; and other useful flavoring agents including aldehydes and esters such as cinnamyl acetate, cinnamaldehyde, eugenyl formate, p-methylanisol, acetaldehyde, benzaldehyde, anisic aldehyde, citral, neral, devanal, vanillin, tolyl aldehyde, 2,6-dimethyloctanal, and 2-ethyl butyraldehyde. In addition, it is intended that the term solvent include volatile fragrances such as natural products and perfume oils. Some representative natural products and perfume oils are ambergris, benzoin, civet, clove, leaf oil jasmine, mate, mimosa, musk, myrrh, orris, sandalwood oil and vetivert oil; aroma chemicals such as amyl salicylate, amyl cinnamic aldehyde, benzyl acetate, citronellol, coumarin, geraniol, isobornyl acetate, ambrette, and terpinyl acetate; and the various classic family perfume oils such as the flora bouquet family, the oriental family, the chypre family, the woody family, the citrus family, the canoe family, the leather family, the spice family, and the herbal family.

In certain embodiments, the compositions further comprise: at least one antimicrobial agent. Suitable representative antimicrobial agents include, but are not limited to,

organic antimicrobial agents (such as benzalkonium chloride),

silicon-containing antimicrobial agents (such as compounds of the formula:

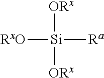

wherein:
R^{x} is H or C₁-C₄alkyl; and
R^{a} is —(C₃-C₆)alkylenyl-dimethyl-(C₆-C₂₂alkyl) quaternary ammonium chloride (especially Aegis 5700 or 5772 commercially available from Aegis) or —(C₃-C₆)alkylenyl-methyl-phenyl-(C₆-C₂₂alkyl) quaternary ammonium chloride (which may be prepared by N-alkylation of N-hexylaniline in a two-step process where N-hexylanlysis is reacted with 3-chloropropyl)triethoxysilane to yield a tertiary amine which then is further quarternized in the second step by reacting with iodomethane, such as described in Saif, et al., Langmuir, 2009, 25, 377-379);
a particulate metal (such as silver);
a salt; and
mixtures thereof.
   3-(Trimethoxysilyl) propyldimethyloctadecyl ammonium chloride is especially preferred.

In certain embodiments, the compositions further comprise: at least one sterilant. Suitable representative sterilants include, but are not limited to, iodine, bromine, chlorine, methanol, ethanol, n-propanol, isopropanol, and mixtures thereof.

In certain embodiments, the compositions further comprise: at least one topical medicament. Suitable representative topical medicaments include, but are not limited to, antibiotics (including triple antibiotics).

In certain embodiments, the compositions further comprise: at least one analgesic. Suitable representative analgesics include, but are not limited to, capsaicin, methyl salicylate, menthol, triethanolamine salicylate, or a mixture thereof.

In certain embodiments, the compositions further comprise: at least one essential oil. Suitable representative essential oils include, but are not limited to, natural mosquito repellent oil, citronella oil, and combinations thereof.

In certain embodiments, the compositions further comprise: at least one preservative.

In certain embodiments, the compositions further comprise: at least one colorant.

In certain embodiments, the compositions further comprise: at least one fragrance.

In certain embodiments, said siloxane copolymer is dimethicone crosspolymer

In certain embodiments, said first solvent, said first additional solvent, and/or said second additional solvent are selected independently from methylsiloxane cyclics having 4 and 5 silicon atoms (such as cyclopentasiloxane), a trimethylsiloxy end-blocked polydimethylsiloxane, ester of aromatic alkoxylated alcohol and fatty carboxylic acid, alkoxylated derivatives of benzyl alcohol (such as PPG-3 benzyl ether ethyl hexanoate), and mixtures thereof.

In certain embodiments, the compositions, comprise:
dimethicone crosspolymer;
cyclopentasiloxane;
PPG-3 benzyl ether ethyl hexanoate;
octadecyldimethyl trimethoxysilylpropyl ammonium chloride; and
benzalkonium chloride.

In certain embodiments, the invention is directed to products comprising the compositions described therein. In certain embodiments, the product is in the form of a gel. In certain embodiments, said product is an selected from the group consisting of: hand sanitizer, antiperspirant, deodorant, skin cream, skin care lotion, moisturizer, acne remover, wrinkle remover, facial cleanser, bath oil, perfume, cologne, sachet, sunscreen, pre-shave lotion, after-shave lotions, liquid soap, shaving soap, shaving lather, shaving gel, hair shampoo, hair conditioner, hair spray, mousse, permanent, depilatory, cuticle coat, make-up, color, cosmetic, foundation, blush, lipstick, lip balm, eyeliner, mascara, oil remover, cosmetic remover, delivery system for oil and water soluble substance, powder, and mixtures thereof.

In certain embodiments, the invention is directed to wound dressing, comprising:
a substrate; and
a composition described herein in contact with said substrate.
   In certain embodiments, the wound dressing further comprises at least one hemostatic agent, such as those described in U.S. Pat. No. 7,604,819, the entire disclosure of which is incorporated herein by reference, as well as, chitosan, zeolites, desmopressin, coagulation factor products (such as prothrombin complex concentrate, cryoprecipitate, fresh frozen plasma), recombinant activated human factor VII, and the like.

In another embodiment, the invention is directed to methods of preparing a siloxane polymer thixotropic base, comprising:
reacting to form a copolymer:
i. a ≡Si—H containing polysiloxane selected from the group consisting of:
   (a) R₃SiO(R₂SiO)ₐ(R″HSiO)_{b}SiR₃;
   (b) HR₂SiO(R′₂SiO)_{c}SiR₂H; and
   (c) HR₂SiO(R₂SiO)ₐ(R″HSiO)_{b}SiR₂H;
      wherein:
      R, R′, R″ are, each independently, C₁-C₆alkyl;
      a is about 0 to about 250;
      b is about 1 to about 250; and
      c is about 0 to about 250; and
ii. an α,ω-diene having the general formula:

CH₂═CH(CH₂)ₓCH═CH₂
wherein:
   x is about 1 to about 20;
wherein said reaction is carried out in the presence of a Noble metal catalyst and a first solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom; and
wherein said reaction is continued until a gel is formed by crosslinking and addition of said ≡Si—H across double bonds in said alpha, omega-diene;
adding a first additional solvent to said gel and subjecting said first additional solvent and said gel to shear force until a siloxane paste is formed;
wherein said first additional solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom;
adding a second additional solvent to said siloxane paste and blending said second additional solvent and said siloxane paste by mixing until said siloxane polymer thixotropic base is formed;
wherein said second additional solvent selected from the group consisting of:
   i. organic compounds;
   ii. compounds containing a silicon atom;
   iii. mixtures of organic compounds;
   iv. mixtures of compounds containing a silicon atom; and
   v. mixtures of organic compounds and compounds containing a silicon atom.

In certain embodiments, the method further comprises:

adding to said siloxane gel, said siloxane paste, or said siloxane polymer thixotropic base at least one component selected from the group consisting of: antimicrobial agent, sterilant, medicament, antibiotic, analgesic, essential oil, preservative, colorant, fragrance, and mixtures thereof.

Carrying out of the process is simply a matter of combining the ≡Si—H containing polysiloxane, the alpha, omega-diene, the low molecular weight silicone oil or other solvent, and the catalyst; mixing these ingredients at room temperature until a gel is formed. Higher temperatures to speed up the process can be used, if desired.

First additional amounts of the low molecular weight silicone oil or solvent are then added to the gel, and the resulting mixture is subjected to shear force to form the paste.

Any type of mixing and shearing equipment may be used to perform these steps such as a batch mixer, planetary mixer, single or multiple screw extruder, dynamic or static mixer, colloid mill, homogenizer, sonolator, or a combination thereof. The low molecular weight silicone oil or other solvent is present at levels generally within the range of about 65% by weight to about 98% by weight, based on the total weight of the composition, preferably about 80% by weight to about 98% by weight, based on the total weight of the composition.

To prepare the siloxane polymer thixotropic base, a second addition of a solvent is added to the paste described just supra.

The solvents useful for this addition include organic compounds, compounds containing a silicon atom, mixtures of organic compounds, mixtures of compounds containing a silicon atom, or mixtures of organic compounds and compounds containing a silicon atom, used on an industrial scale to dissolve, suspend, or change the physical properties of other materials.

Preferred for the second solvent are low molecular weight silicone oils, and as set forth supra, it is intended herein to include low molecular weight linear and cyclic volatile methyl siloxanes, low molecular weight linear and cyclic volatile and non-volatile alkyl and aryl siloxanes, and low molecular weight linear and cyclic functional siloxanes.

Also preferred for the second solvent are organic compounds such as, for example, esters of aromatic alkoxylated alcohols and fatty carboxylic acids having the general formula R′0-[Alk-O]—R″, wherein R′ is an organic moiety that contains at least one aromatic nucleus RN; R″ is a fatty alkyl group; and —[Alk-O-] is an alkoxy spacer, preferably C₁-C₂₀alkyoxy spacer.

The aromatic nucleus RN may contain from 6 to 20 carbon atoms exclusive of substitution. Nuclei RN having 6 to 10 carbon atoms are preferred. Non-limiting examples of the aromatic nuclei RN are benzene, naphthalene and anthracene nuclei, which contain 6, 10, and 14 carbon atoms, respectively. The preferred method for combining the second solvent into the paste is simple blending rather than shear mixing.

The siloxane polymer thixotropic base has been found useful as a carrier for a multitude of actives that are beneficial as cosmetics; pharmaceuticals; antimicrobial, antifungal, antialgae agents; medicaments; bug repellents; coagulants or hemostatic agent; and the like. It has been found that minor variations in the amount of and type of the second solvent used to make the polysiloxane polymer thixotropic base have a significant impact on the material being added to the base, in that, many different types of materials can be added to the thixotropic base and remain compatible.

In certain embodiments, the invention is directed to methods of inhibiting microbes on skin of a user, comprising: applying a composition comprising at least one antimicrobial agent onto said skin of said user. In certain embodiments, the method further comprises washing said skin of said user at least once. In certain embodiments of the method, the composition is applied to at least one hand of said user; and the user wears a glove on said at least one hand.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

Samples of the composition of this invention were prepared by blending components A or B with both C and D. All of the weights are in grams.

The samples were then tested. The test subjects were Caucasian females. About 1.0 g of each composition was applied to the back of the hand of the test subject. All of the comments were obtained within 60 seconds after application and evaluation of each composition.

The compositions and comments from the test subjects are shown in Table I.

TABLE I

| Materials | Sample 1 | Sample 2 |
|---|---|---|
| A | 40 | |
| B | | 40 |
| C | 1.6 | 1.5 |
| D | 1.2 | 0.7 |
| Comments | Slick feel, oily | Grabby feel, |
| | feel | wet feel |

A = Dow Corning 9040 elastomer blend (a mixture of high molecular weight silicone elastomers (dimethicone crosspolymer) in cyclopentasiloxane
B = Dow Corning 9045 elastomer blend (a mixture of high molecular weight silicone elastomers (dimethicone crosspolymer) in cyclopentasiloxane
C = a mixture of methylsiloxane cyclics having 4 and 5 silicon atoms, respectively
D = a trimethylsiloxy end-blocked polydimethylsiloxane having 100 cS viscosity

### Example 2

Samples were prepared as shown in Example 1. The compositions and comments from the test subjects are shown in Table II.

TABLE II

| | Sample 1 | Sample 2 | Sample 3 | Sample 4 | Sample 5 | Sample 6 | Sample 7 |
|---|---|---|---|---|---|---|---|
| Materials | Components (in grams) | | | | | | |
| A | 15 | 15 | 15 | 15 | 15 | 15 | 15 |
| C | 1.69 | | | | 0.55 | 0.2 | 3 |
| E | | 1 | | | | 1 | |
| D | | | 0.25 | | | | |
| F | | | | 0.35 | | | |
| G | 0.00015 | 0.00015 | 0.00015 | 0.00015 | 0.00015 | 0.00015 | 0.00015 |
| Tester | Ratings (1 = best feel; 10 = worst feel) | | | | | | |
| 1 | 5 | | 10 | | 1 | | |
| 2 | | | | 1 | 1 | | |
| 3 | 5 | 5 | 1 | 1 | 1 | 10 | 10 |
| 4 | 3 | 5 | 3 | 1 | 1 | 3 | 10 |
| 5 | 1 | | | | | | 10 |
| Comments | Slippery, | Balls up, | Balls up, | Slick feel, | Slick feel, | Oily | Very |
| | wet feel | does not | does not | sticky feel | spreads | | slippery, |
| | | spread | spread | | | | oily |

A = Dow Corning 9040 elastomer blend (a mixture of high molecular weight silicone elastomers (dimethicone crosspolymer) in cyclopentasiloxane
C = a mixture of methylsiloxane cyclics having 4 and 5 silicon atoms, respectively
D = a trimethylsiloxy end-blocked polydimethylsiloxane having 100 cS viscosity
E = a trimethylsiloxy end-blocked polydimethylsiloxane having 50 cS viscosity
F = a trimethylsiloxy end-blocked polydimethylsiloxane having 1000 cS viscosity
G = preservative

### Example 3

Samples were prepared as shown in Example 1. The compositions and comments from the test subjects are shown in Table III.

TABLE III

| Materials | Sample 1 | Sample 2 | Sample 3 | Sample 4 |
|---|---|---|---|---|
| A | 3000 | 94.047 | 94.047 | 94.047 |
| C | 111 | | | |
| H | | 5.416 | 5.416 | 5.416 |
| G | 0.5 | 0.0009 | 0.0009 | 0.0009 |
| I | | 0.527 | 1.54 | 0.263 |
| Comments | Slick feel, | Slick feel, | Slick feel, | Slick feel, |
| | spreads | spreads | spreads | spreads easily |

A = Dow Corning 9040 elastomer blend (a mixture of high molecular weight silicone elastomers (dimethicone crosspolymer) in cyclopentasiloxane
C = a mixture of methylsiloxane cyclics having 4 and 5 silicon atoms, respectively
G = preservative
H = Crodamol SFX ester of aromatic alkoxylated alcohol and fatty carboxylic acid
I = (CH₃O)₃Si(CH₂)₃N⁺ dimethyloctadecyl chloride

### Example 4

This example tests the compositions of the invention (in the form of a gel) in killing or reducing E. coli that has be inoculated into the gel containing the compositions of the invention.

### Method 1:

The formulations were prepared, one with the antimicrobial and one without the antimicrobial as a control. A saline solution with approximately 1000 CFU/ml E. coli was prepared and used as the inoculum. The formulation was placed in resealable plastic bags and 3 ml of the inoculum added to each. Also, a separate bag with 3 ml of only inoculum was used as a positive control.

The E. coli used, purchased from Quanti-Cult™, was derived from original ATCC stock cultures. They were received dehydrated. They are then rehydrated, and transferred to a sterile Erlenmeyer flask containing 100 ml of tryptic soy broth and incubated overnight at 35° C. The cultures were only used for testing on that day. A 1 ml aliquot of that culture was put into a clean flask with fresh broth daily.

The formulations were exposed for 60 minutes. Since the formulation is very hydrophobic, the bags were kneaded every 10 minutes to ensure contact of the inoculum with the material. At the end of the exposure period, 5 grams of material was placed in petri dishes (in triplicate), spread with a sterile cotton swab and then covered with tryptic soy agar and incubated for 24 hours at 35° C. At the end of the incubation period colony counts were compared. The results are shown below.

### Method 2:

The compositions were prepared as above and the same method used. However, five exposure periods were investigated (60 seconds, 60 minutes, 4 hours, 8 hours and 24 hours). Also, at the end of the exposure periods, rather than spreading the composition in petri dishes, only the inoculum that separated from the gel in the releasable plastic bag was incubated and tested for bacteria. Equal volumes of the inoculum was withdrawn carefully from each of the bags and covered with tryptic soy agar as above, incubated for 24 hours at 35° C. and colony counts compared at the end of the incubation period.

When an appropriate exposure period was determined, the experiment was repeated using various levels of bacteria.

Control samples were also evaluated. This included gel without any antimicrobial agent (designated as “Comparative”) and a control utilizing only the releasable plastic bag (designated as “Control”).

### Study 1 Exposure Time

This study evaluated the length of time for the effectiveness of the gel of the invention to eliminate or reduce the amount of bacteria. A 3-8 ml incubated 3000 CFU/ml E. coli sample was introduced into a releasable plastic bag containing the gel of the invention for increasing times of exposure. The times of exposure were:
60 seconds
60 minutes
4 hours
8 hours
24 hours
   After which the sample of E. coli were incubated and the colony count were performed. Each sample was run in triplicate for a total of 18 samples.

### Study 2 Concentration Study

This study evaluated the effectiveness of gel of the invention to eliminate or reduce the amount of increasing amounts of bacteria. A 3-8 ml E. coli sample containing increasing amounts of E. coli was introduced into a releasable plastic bag containing gel of the invention. The colony counts were:
0 CFU/ml
1000 CFU/ml
3000 CFU/ml
6000 CFU/ml
   Each sample was exposed to the gel of the invention for a specific time based on the effectiveness in Study 1. After which the sample of E. coli was incubated and the colony count was performed. Each sample was run in triplicate for a total of 12 samples.

### Results

### Method 1:

| Sample | Results |
|---|---|
| Inoculums alone in releasable plastic bag | Too numerous to count |
| (control) | |
| Gel (with antimicrobial) | Average 40 CFU/plate |
| Gel (without antimicrobial) (comparative) | Average 91 CFU/plate |

### Example 5

The routine use of lotions to minimize the development of dermatitis on hands of health professionals that undergo many wash procedures per day has been a component of infection control recommendations for many years. In the past, the primary role for these lotions was to lubricate epithelial tissue and replace emollients that were removed. The purpose of this example was to examine antimicrobial properties of a hand sanitizer of the invention (containing dimethicone crosspolymer; cyclopentasiloxane; PPG-3 benzyl ether ethyl hexanoate; octadecyldimethyl trimethoxysilylpropyl ammonium chloride; and benzalkonium chloride) using the gram-negative bacillus Serratia marcescens as an indicator microorganism for effectiveness.

Experiments were developed to initially determine the antimicrobial activity following bacterial challenge on hands coated with the hand gel. The product was also tested for extended effectiveness during prolonged glove use by volunteers. Finally, the ability of hand sanitizer to exhibit a demonstrable antibacterial effect following repeated hand washing was investigated.

### Experimental Design:

Phase 1:

Hands of potential volunteers for the investigation were initially screened for any observable skin conditions or injuries (i.e., broken skin) that could prevent them from participation in the project. The condition of the volunteers' hands was also visually evaluated throughout the three phases of the study. Four (4) volunteers were then instructed to wash their hands with a non-antimicrobial liquid soap and water for one minute to initially cleanse the skin before application of the experimental gel. A small amount (dime size) of hand sanitizer was then applied onto the participants' hands and rubbed vigorously into the skin. Immediately after this application, a 2×2″ square section of the dorsal surface of each hand was swabbed with a 1:100,000 dilution (left hand) and a 1:1,000,000 dilution (right hand) of a 24-hour broth culture of the red-pigmented bacterial species, S. marcescens. These two bacterial dilutions were used following multiple preliminary experiments that were carried out to determine concentrations of S. marscecens that would reproducibly challenge hand gel activity. The resultant concentrations of S. marscecens used to challenge hands were approximately 1×105 bacteria/mL (left hand) and 1×104 bacteria/mL (right hand), respectively. Applied bacterial suspensions were allowed to dry before proceeding with further testing. Subsequently, antibacterial activity of hand sanitizer as determined by the presence of S. marcescens at the application sites was assayed by swabbing the 2×2″ test areas with sterile cotton swabs moistened in sterile trypticase soy broth. Sample intervals of 1 minute, 30 minutes, 2, 4, 6, and 8 hours were utilized. Collected samples were streaked onto trypicase soy agar plates and incubated aerobically at room temperature for 48-72 hours. Incubation of S. marcescens under these conditions allowed for both microbial growth and red pigment production by the bacteria.

Phase 2:

Four volunteers initially prepared their hands as described above. Hand sanitizer was applied to both hands and allowed it to dry, followed by application of the 1:100,000 and 1:1,000,000 dilutions of cultured S. marcescens. Hands were allowed to dry and zero minute control samples were taken before proceeding. Participants then washed their hands for 15 seconds with a non-antimicrobial soap after sampling at designated periods of 0 (control) minutes, 30 minutes, 2, 4, 6 and 8 hours. Samples were taken from the S. marcescens-seeded hand sites using sterile broth moistened cotton swabs, cultured on trypticase soy agar plates, incubated, and observed for bacterial growth as described above.

Phase 3:

The participants washed their hands as described above prior to the glove use portion of the investigation, followed by application of hand sanitizer. The S. marscecens test dilutions were swabbed onto the right and left hand test sites, respectively. Participants then donned nitrile gloves and continued to wear them for one hour while performing routine work tasks. At the end of this interval gloves were removed, samples collected from test sites using moistened cotton swabs, and trypticase soy agar plates seeded with material from the skin. Prepared culture plates were incubated at room temperature for 48-72 hours prior to observation for red-pigmented S. marcescens colonies.

### Results:

Phase 1:

Sixteen (16) test samples from each S. marcescens dilution (1:100,000 and 1:1,000,000) were assayed. Culture findings are presented in Table 1. All S. marcescens control cultures collected without exposure to the hand gel yielded confluent bacterial growth. With regard to the 1-minute samples, 25/32 cultures were positive for red-pigmented colonies. These showed higher levels of bacterial growth on each plate than was detected for later experimental collection intervals. In most cases the pigmented growth for the one minute-exposure plates was similar in intensity per plate to that noted for the positive control plates, or was slightly less. For all other test cultures where positive growth was observed, the presence of bacteria cultured for these intervals was considerably less. It was interesting to note that as the time intervals of skin exposure to hand sanitizer increased, the resultant detection of S. marcescens colonies decreased, until by six hours none of the test cultures yielded red-pigmented colonies (0/32). No hand dermatitis problems were noted for any of the volunteers.

TABLE 1
Effect of Hand Sanitizer with Repeated Bacterial Exposure

| Sample Time | 1:100,000 dilution | 1:1,000,000 dilution |
|---|---|---|
| Control plates | 18/18 | 18/18 |
| 0 minutes | 17/18 | 5/18 |
| 30 minutes | 3/18 | 0/18 |
| 2 hours | 1/18 | 0/18 |
| 4 hours | 0/18 | 0/18 |
| 6 hours | 0/18 | 0/18 |
| 8 hours | 0/18 | 0/18 |

Phase 2:

Eighteen (18) test samples of each dilution were completed for Phase 2 and the observed results are presented in Table 2. The zero-minute time interval represented the only sampling before any hand washing had occurred. The observed results were comparable to the one-minute data obtained in Phase 1, in that all test samples produced positive results but the per plate quantity of growth was much less than that observed on the corresponding positive controls. All other positive test cultures observed at later time intervals produced considerably less pigmented growth, much like what was seen for cultures in Phase 1. No hand dermatitis problems were noted for any of the volunteers.

TABLE 2
Effect of Hand Gel with Repeated Handwashing and Bacterial Exposure

| Sample Time | 1:100,000 dilution | 1:1,000,000 dilution |
|---|---|---|
| Control plates | 18/18 | 18/18 |
| 0 minutes | 17/18 | 5/18 |
| 30 minutes | 3/18 | 0/18 |
| 2 hours | 1/18 | 0/18 |
| 4 hours | 0/18 | 0/18 |
| 6 hours | 0/18 | 0/18 |
| 8 hours | 0/18 | 0/18 |

Phase 3:

All except one (15/16) experimental cultures from samples taken after removal of gloves were negative for red-pigmented bacteria (Table 3). In contrast, in a separate positive control assay, a participant applied bacteria only and wore gloves for one hour without the use of hand sanitizer. Resultant cultures from control test sites were positive (4/4). The qualitative presence of S. marcescens in these controls was much less than that found for control plates using diluted bacteria only (16/16). No hand dermatitis problems were noted for any of the volunteers.

TABLE 3
Effect of Hand Gel during Glove Use

| Sample Time | 1:100,000 dilution | 1:1,000,000 dilution |
|---|---|---|
| Control plates | 16/16 | 16/16 |
| 1 hour | 1/16 | 0/16 |

### Summary:

The example demonstrated that the hand sanitizer of the invention provided an antimicrobial benefit. Laboratory testing with S. marcescens demonstrated that the gel was effective in inhibiting test bacteria applied onto the skin during multiple hand wash procedures and prolonged wearing of gloves. Additionally, none of the participating volunteers demonstrated any irritation dermatitis symptoms with exposure to the hand sanitizer.

When ranges are used herein for physical properties, such as molecular weight, or chemical properties, such as chemical formulae, all combinations, and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A composition, comprising:
A. a siloxane gel comprising:
a. a first solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom;
b. a siloxane copolymer comprising a reaction product of:
i. an hydridopolysiloxane selected from the group consisting of:
(a) R₃SiO(R′₂SiO)ₐ(R″HSiO)_{b}SiR₃;
(b) HR₂SiO(R′₂SiO)_{c}SiR₂H; and
(c) HR₂SiO(R′₂SiO)ₐ(R″HSiO)_{b}SiR₂H;
wherein:
R, R′, R″ are, each independently, C₁-C₆alkyl;
a is about 0 to about 250;
b is about 1 to about 250; and
c is about 0 to about 250; and
ii. an α,ω-diene having the general formula:
CH₂═CH(CH₂)ₓCH═CH₂
wherein:
x is about 1 to about 20;
B. a first additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom;
C. a second additional solvent selected from the group consisting of:
a. organic compounds;
b. compounds containing a silicon atom;
c. mixtures of organic compounds;
d. mixtures of compounds containing a silicon atom; and
e. mixtures of organic compounds and compounds containing a silicon atom.

2. A composition of claim 1, further comprising:
at least one antimicrobial agent.

3. A composition of claim 2,
wherein said antimicrobial agent is an organic antimicrobial agent.

4. A composition of claim 2,
wherein said at least one antimicrobial agent is benzalkonium chloride.

5. A composition of claim 2,
wherein said at least one antimicrobial agent is a silicon-containing antimicrobial agent.

6. A composition of claim 2,
wherein said at least one antimicrobial agent is a compound of the formula: 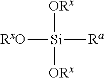
wherein:
R^{x} is H or C₁-C₄alkyl; and
R^{a} is —(C₃-C₆)alkylenyl-dimethyl-(C₆-C₂₂alkyl) quaternary ammonium chloride or —(C₃-C₆)alkylenyl-methyl-phenyl-(C₆-C₂₂alkyl) quaternary ammonium chloride.

7. A composition of claim 2,
wherein said at least one antimicrobial agent is 3-(trimethoxysilyl) propyldimethyloctadecyl ammonium chloride.

8. A composition of claim 2,
wherein said antimicrobial agent is a particulate metal.

9. A composition of claim 2,
wherein said antimicrobial agent is a salt.

10. A composition of claim 1, further comprising:
at least one sterilant.

11. A composition of claim 10,
wherein said sterilant is selected from the group consisting of: iodine, bromine, chlorine, methanol, ethanol, n-propanol, isopropanol, and mixtures thereof.

12. A composition of claim 1, further comprising:
at least one topical medicament.

13. A composition of claim 12,
wherein said topical medicament is an antibiotic.

14. A composition of claim 13,
wherein said antibiotic is a triple antibiotic.

15. A composition of claim 1, further comprising:
at least one analgesic.

16. A composition of claim 15,
wherein said analgesic is capsaicin, methyl salicylate, menthol, triethanolamine salicylate, or a mixture thereof.

17. A composition of claim 1, further comprising:
at least one essential oil.

18. A composition of claim 17,
wherein the at least one essential oil is a natural mosquito repellent oil, citronella oil, or a combination thereof.

19. A composition of claim 1, further comprising:
at least one preservative.

20. A composition of claim 1, further comprising:
at least one colorant.

21. A composition of claim 1, further comprising:
at least one fragrance.

22. A composition of claim 1,
wherein said siloxane copolymer is dimethicone crosspolymer.

23. A composition of claim 1,
wherein said first solvent is selected from the group consisting of: methylsiloxane cyclics having 4 and 5 silicon atoms, a trimethylsiloxy end-blocked polydimethylsiloxane, ester of aromatic alkoxylated alcohol and fatty carboxylic acid, alkoxylated derivatives of benzyl alcohol, and mixtures thereof.

24. A composition of claim 1,
wherein said first additional solvent is selected from the group consisting of: methylsiloxane cyclics having 4 and 5 silicon atoms, a trimethylsiloxy end-blocked polydimethylsiloxane, ester of aromatic alkoxylated alcohol and fatty carboxylic acid, alkoxylated derivatives of benzyl alcohol, and mixtures thereof.

25. A composition of claim 1,
wherein said second additional solvent is selected from the group consisting of: methylsiloxane cyclics having 4 and 5 silicon atoms, a trimethylsiloxy end-blocked polydimethylsiloxane, ester of aromatic alkoxylated alcohol and fatty carboxylic acid, alkoxylated derivatives of benzyl alcohol, and mixtures thereof.

26. A composition, comprising:
dimethicone crosspolymer;
cyclopentasiloxane;
PPG-3 benzyl ether ethyl hexanoate;
octadecyldimethyl trimethoxysilylpropyl ammonium chloride; and
benzalkonium chloride.

27. A product, comprising:
said composition of claim 1.

28. A product of claim 27,
wherein said product is in the form of a gel.

29. A product of claim 27,
wherein said product is an selected from the group consisting of: hand sanitizer, antiperspirant, deodorant, skin cream, skin care lotion, moisturizer, acne remover, wrinkle remover, facial cleanser, bath oil, perfume, cologne, sachet, sunscreen, pre-shave lotion, after-shave lotions, liquid soap, shaving soap, shaving lather, shaving gel, hair shampoo, hair conditioner, hair spray, mousse, permanent, depilatory, cuticle coat, make-up, color, cosmetic, foundation, blush, lipstick, lip balm, eyeliner, mascara, oil remover, cosmetic remover, delivery system for oil and water soluble substance, powder, and mixtures thereof.

30. A hand sanitizer, comprising:
a composition of claim 1.

31. A wound dressing, comprising:
a substrate; and
a composition of claim 1 in contact with said substrate.

32. A wound dressing of claim 31, further comprising:
at least one hemostatic agent.

33. A method of preparing a siloxane polymer thixotropic base, comprising:
reacting to form a copolymer:
i. a Si—H containing polysiloxane selected from the group consisting of:
(a) R₃SiO(R′₂SiO)ₐ(R″HSiO)_{b}SiR₃;
(b) HR₂SiO(R′₂SiO)_{c}SiR₂H; and
(c) HR₂SiO(R′₂SiO)ₐ(R″HSiO)_{b}SiR₂H;
wherein:
R, R′, R″ are, each independently, C₁-C₆alkyl;
a is about 0 to about 250;
b is about 1 to about 250; and
c is about 0 to about 250; and
ii. an α,ω-diene having the general formula:
CH₂═CH(CH₂)ₓCH═CH₂
wherein:
x is about 1 to about 20;
wherein said reaction is carried out in the presence of a Noble metal catalyst and a first solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom; and
wherein said reaction is continued until a gel is formed by crosslinking and addition of said Si—H across double bonds in said alpha, omega-diene;
adding a first additional solvent to said gel and subjecting said first additional solvent and said gel to shear force until a siloxane paste is formed;
wherein said first additional solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom;
adding a second additional solvent to said siloxane paste and blending said second additional solvent and said siloxane paste by mixing until said siloxane polymer thixotropic base is formed;
wherein said second additional solvent selected from the group consisting of:
i. organic compounds;
ii. compounds containing a silicon atom;
iii. mixtures of organic compounds;
iv. mixtures of compounds containing a silicon atom; and
v. mixtures of organic compounds and compounds containing a silicon atom.

34. A method of claim 33, further comprising:
adding to said siloxane gel, said siloxane paste, or said siloxane polymer thixotropic base at least one component selected from the group consisting of:
antimicrobial agent, sterilant, medicament, antibiotic, analgesic, essential oil, preservative, colorant, fragrance, and mixtures thereof.

35. A method of inhibiting microbes on skin of a user, comprising:
applying a composition of claim 2 onto said skin of said user.

36. A method of inhibiting microbes on skin of a user of claim 35, further comprising:
washing said skin of said user at least once.

37. A method of inhibiting microbes on skin of a user of claim 35,
wherein said composition is applied to at least one hand of said user; and
wherein said user wears a glove on said at least one hand.

38. A product, comprising:
said composition of claim 2.

39. A product, comprising:
said composition of claim 26.

40. A hand sanitizer, comprising:
a composition of claim 2.

41. A wound dressing, comprising:
a substrate; and
a composition of claim 2 in contact with said substrate.

42. A hand sanitizer, comprising:
a composition of claim 26.

43. A wound dressing, comprising:
a substrate; and
a composition of claim 26 in contact with said substrate.
